(19) United States
(12) Patent Application Publication
(10) Pub. No.: US 2011/0190253 A1
(43) Pub. Date: Aug. 4, 2011

(54) METHOD FOR TREATING TUBERCULOSIS

(76) Inventor: John S. Blanchard, New Rochelle, NY (US)

(21) Appl. No.: 12/736,906

(22) PCT Filed: May 15, 2009
(86) PCT No.: PCT/US2009/003057

§ 371 Date: Mar. 10, 2011

Related U.S. Application Data
(60) Provisional application No. 61/128,933, filed on May 27, 2008.

(51) Int. Cl. A61K 31/546; A61K 31/397; A61P 31/06

(52) U.S. Cl. 514/202; 514/210.06

(57) ABSTRACT

The present invention generally relates to methods for treating tuberculosis in a subject comprising administering to the subject an antibiotic in conjunction with clavulanic acid or salt thereof. The antibiotic can be carbapenem (e.g., meropenem or imipenem) or cefuroxime. The present invention also relates to related pharmaceutical compositions and methods for manufacturing said pharmaceutical compositions.

### CROSS-REFERENCE TO RELATED APPLICATION

This application claims the benefit of U.S. Provisional Patent Application No. 61/128,933, filed May 27, 2008, the content of which is hereby incorporated by reference into the subject application.

### STATEMENT OF GOVERNMENT SUPPORT

The invention disclosed herein was made with U.S. Government support under Grant Number AI33696 from The National Institutes of Health. Accordingly, the U.S. Government has certain rights in this invention.

### FIELD OF THE INVENTION

The present invention generally relates to methods for treating tuberculosis in subject comprising administering an antibiotic and clavulanic acid and related pharmaceutical compositions and methods of production and use.

### BACKGROUND OF THE INVENTION

Throughout this application various publications are referred to by Arabic numerals in parentheses. Full citations for these references may be found at the end of the specification immediately preceding the claims. The disclosures of these publications are hereby incorporated by reference in their entireties into the subject application to more fully describe the art to which the subject application pertains.

In recent years, multi-drug resistant (MDR) tuberculosis as well as extremely drug-resistant (XDR) tuberculosis have evolved, threatening our ability to clinically treat this deadly human pathogen (1). Hence, it is imperative that new drugs and treatment paradigms be made available to curb this crisis. Historically, one of the most effective therapeutic classes of antibacterials have been the structural β-lactam ring class of antibiotics, all of which contain the structural β-lactam ring motif. This class of antibacterial compounds inhibits the bacterial D, D-transpeptidases (2), which catalyze the final step of peptidoglycan cross-linking. This cross-linking activity is essential for cell-wall maturation and cell survival.

Resistance to this class of antibiotics in M. tuberculosis arises from a chromosomally encoded Ambler class A β-lactamase, BlaC (3), which catalyzes the hydrolysis of the β-lactam antibiotic ring. The catalytic mechanism of BlaC involved (1) the activation of an active-site nucleophile, Ser70 by Lys73 and/or Glu 166, (2) attack on the β-lactam ring carbonyl with the formation of a covalent acyl-enzyme complex, and (3) hydrolysis of the ester bond via a conserved active-site water and Glu166, forming the free enzyme and the ring-opened product (4-8). Because of the intrinsic resistance as a result of the constitutive production of the β-lactamase, β-lactams have never been systematically applied with success to the treatment of TB infections. Current treatment instead relies on a regiment of four compounds (isoniazid, rifampicin, ethambutol, and pyrazinamide) co-administered over a 6-month period.

Accordingly, there is a compelling need to develop more effective methods of treatment for tuberculosis, especially for treatment of MDR tuberculosis and XDR tuberculosis. The present invention satisfies that need.

### SUMMARY OF THE INVENTION

The present invention is directed to a method for treating tuberculosis in a subject comprising administering to the subject an amount of carbapenem and clavulanic acid (or salt thereof) effective to treat tuberculosis. The present invention is also directed to a method for treating tuberculosis in a subject comprising administering to the subject an amount of cefuroxime and clavulanic acid (or salt thereof) effective to treat tuberculosis. The present invention is further directed to pharmaceutical compositions comprising carbapenem and clavulanic acid (or salt thereof) or cefuroxime and clavulanic acid (or salt thereof). The present invention is also directed to methods of producing these pharmaceutical compositions and their use in treating tuberculosis.

### BRIEF DESCRIPTION OF THE FIGURES

FIG. 1. In vitro inhibition of tuberculosis. 96 well plates were plated with the Erdman strain of M. tuberculosis. The plates were then contacted with amoxicillin, cefuroxime, imipenem or meropenem. As indicated in FIG. 1, the first 4 columns were also contacted with clavulanate (0-2.0 ug/ml). Following a one-week incubation period, inhibition of M. tuberculosis growth was examined.

FIG. 2. M. tuberculosis growth following one week period of incubation. Antibiotics were administered on day 1 of the one-week incubation period. Concentrations of antibiotic and clavulanate are indicated.

FIGS. 3 and 4. M. tuberculosis growth following one week period of incubation. Antibiotics were administered on days 1 and 4 of the one-week incubation period. Concentrations of antibiotic and clavulanate are indicated.

### DETAILED DESCRIPTION OF THE INVENTION

The present invention provides a method for treating tuberculosis (TB) in a subject comprising administering to the subject an amount of carbapenem and clavulanic acid (or salt thereof) effective to treat tuberculosis. The present invention further provides a method for treating tuberculosis in a subject comprising administering to the subject an amount of cefuroxime and clavulanic acid (or salt thereof) effective to treat tuberculosis.

In the preferred embodiment of the invention, the tuberculosis is multidrug-resistant tuberculosis (MDR tuberculosis) or extensively drug-resistant tuberculosis (XDR tuberculosis). As used herein, “MDR tuberculosis” shall mean a form of tuberculosis that is resistant to two or more of the primary drugs (isoniazid and rifampicin) used for the treatment of tuberculosis. As used herein, “XDR tuberculosis” shall mean a form of tuberculosis resistant to at least isoniazid and rifampicin among the first-line anti-TB drugs, is resistant to any fluoroquinolone and at least one of three injectable second-line drugs, such as amikacin, kanamycin or capreomycin. In the preferred embodiment, the subject is human.

Numerous carbapenems for use in the present invention are known in the art, and include, but are not limited to meropenem, imipenem, ertapenem, faropenem, doripenem, or panipenem. In the preferred embodiment of the present invention, the carbapenem is meropenem or imipenem. Most preferably, the carbapenem is meropenem.

Clavulanic acid and its salts are beta-lactamase inhibitors and are well known in the art (9, 10, 11, 12). U.S. Pat. Nos. 5,726,170, 6,048,977, 6,051,703, and 5,994,534, which are hereby incorporated by reference in their entirety, disclose salts of clavulanic acid and methods for making same. In the preferred embodiment of the present invention, the clavulanic acid is potassium clavulanate (referred herein as clavulanate), which is the potassium salt of clavulanic acid.

In accordance with the above methods, the carbapenems such as meropenem, imipenem or cefuroxime may be in the same pharmaceutical formulation as clavulanic acid (or salt thereof). Alternatively, the meropenem, imipenem or cefuroxime may be in a different pharmaceutical formulation from clavulanic acid (or salt thereof).

In the preferred embodiment of the invention, the compounds for treating tuberculosis can easily be administered parenterally such as for example, by intramuscular, intrathecal, subcutaneous, intraperitoneal, intravenous bolus injection or intravenous infusion. Parenteral administration can be accomplished by incorporating the compounds of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Additionally, the compounds of the present methods can be designed for oral, nasal, lingual, sublingual, buccal and intrabuccal administration and made without undue experimentation by means well known in the art, for example with an inert diluent or with an edible carrier. The compounds may be enclosed in gelatin capsules or compressed into tablets. For the purpose of oral therapeutic administration, the compounds of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, cornstarch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

Rectal administration includes administering the compounds into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the pharmaceutical composition through the skin. Transdermal formulations include patches (such as the well-known nicotine patch), ointments, creams, gels, salves and the like.

The present invention includes nasally administering to the mammal therapeutically effective amounts of the compounds. As used herein, nasally administering or nasal administration includes administering the compounds to the mucous membranes of the nasal passage or nasal cavity of the patient. As used herein, compounds for nasal administration of a composition include therapeutically effective amounts of the compound prepared by well-known methods to be administered, for example, as a nasal spray, nasal drop, suspension, gel, ointment, cream or powder. Administration of the compounds may also take place using a nasal tampon or nasal sponge.

The compounds used in the present invention are well known in the art, as are their modes of administration. For example, meropenem and clavulanic acid (or salt thereof) can be dissolved in sterile water for intravenous bolus injection. Alternatively, meropenem can be dissolved in Sodium Chloride Injection 0.9% solution for intravenous infusion. In another example, imipenem can be formulated for intravenous infusion by dissolving it in sterile isotonic saline or a 5% dextrose solution. In another example, cefuroxime can be formulated for intravenous infusion by dissolving it with sterile isotonic saline. In the preferred embodiment of the present invention, meropenem, imipenem or cefuroxime, are in the same pharmaceutical formulation as clavulanic acid (or salt thereof), and are administered via intravenous infusion.

In accordance with the present invention, effective amounts of the compounds to be administered can be formulated without undue experimentation for administration to a mammal, including humans, as appropriate for the particular application. Additionally, proper dosages of the compounds can be determined without undue experimentation using standard dose-response protocols.

As used herein, an “amount effective to treat tuberculosis in a subject” or a “therapeutically effective amount” can mean the amount of a compound or compounds effective to ameliorate or eliminate tuberculosis once it has been established or alleviate the characteristic symptoms of tuberculosis. As used herein, these terms also encompass, depending on the condition of the patient, preventing the onset of a disease or condition or symptoms associated with tuberculosis, including reducing the severity of the disease or condition or symptoms associated therewith prior to affliction with said disease or condition. Alternatively, an “amount effective to treat tuberculosis in a subject” or a “therapeutically effective amount” can mean the amount of a compound or compounds effective to reduce or eliminate the presence of Mycobacterium tuberculosis in the subject. An amount effective to treat tuberculosis or a therapeutically effective amount will vary with the age and general condition of the subject, the efficiency of the delivery method (i.e., the percent of the dose that is deposited in the target area), the severity of the condition being treated, the particular compound or composition being administered, the duration of the treatment, the nature of any concurrent treatment, the carrier used, and like factors within the knowledge and expertise of those skilled in the art. As appropriate, an amount effective to treat tuberculosis or a therapeutically effective amount in any individual case can be determined by one of ordinary skill in the art by reference to the pertinent texts and literature and/or by using routine experimentation (see, e.g., Remington, The Science and Practice of Pharmacy (20^{th} ed. 2000)).

In accordance with the present invention, the effective amount of meropenem can be from about 250 mg/day to about 6000 mg/day. In the preferred embodiment of the present invention, the effective amount of meropenem is about 1500 mg/day to about 3000 mg/day.

In accordance with the present invention, the effective amount of imipenem can be from about 250 mg/day to about 6000 mg/day. In the preferred embodiment of the present invention, the effective amount of imipenem is about 500 mg/12 hours to about 1000 mg/12 hours.

In accordance with the present invention, the effective amount of clavulanic acid (or salt thereof) can be from about 25 mg/day to about 1000 mg/day. In the preferred embodiment of the present invention, the effective amount of clavulanic acid (or salt thereof) is about 50 mg/day to about 500 mg/day.

In accordance with the present invention, the effective amount of cefuroxime can be from about 750 mg/day to about 9000 mg/day. In the preferred embodiment of the present invention, the effective amount of cefuroxime is about 2250 mg/day to about 4500 mg/day. In accordance with standard dose-response protocols, the frequency of the dosages can be increased or decreased by adjusting the effective amounts of the compounds being administered at a given time.

The present invention also provides a pharmaceutical composition comprising therapeutically effective amounts of carbapenem and clavulanic acid (or salt thereof), and a pharmaceutically acceptable carrier.

The present invention further provides a pharmaceutical composition comprising therapeutically effective amounts of cefuroxime and clavulanic acid (or salt thereof), and a pharmaceutically acceptable carrier.

As used herein, a “pharmaceutically acceptable” carrier shall mean a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like. In the preferred embodiment, the pharmaceutical composition is formulated for parenteral or oral administration.

The present invention further provides a method for manufacturing a pharmaceutical composition for treating tuberculosis in a subject comprising formulating therapeutically effective amounts of carbapenem and clavulanic acid (or salt thereof) with a pharmaceutically acceptable carrier.

The present invention also provides a method for manufacturing a pharmaceutical composition for treating tuberculosis in a subject comprising formulating therapeutically effective amounts of cefuroxime and clavulanic acid (or salt thereof) with a pharmaceutically acceptable carrier.

When used for treatment methods, the above-described pharmaceutical compositions would be in a pharmaceutically acceptable excipient.

The above-described pharmaceutical compositions can be formulated without undue experimentation for administration to a mammal, including humans, as appropriate for the particular application. Additionally, proper dosages of the compositions can be determined without undue experimentation using standard dose-response protocols.

Additionally, the present invention provides a method for treating tuberculosis, including MDR tuberculosis and XDR tuberculosis, in a subject comprising administering to the subject an amount of penicillin V, ampicillin, ticarcillin, cephadrine, cefaclor or cefixime, and an amount of clavulanic acid (or salt thereof) effective to treat tuberculosis. The present invention also provides a pharmaceutical composition comprising therapeutically effective amounts of penicillin V, ampicillin, ticarcillin, cephadrine, cefaclor or cefixime, and clavulanic acid (or salt thereof), and a pharmaceutically acceptable carrier, and methods for making the pharmaceutical composition.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Example 1

### Materials and Methods

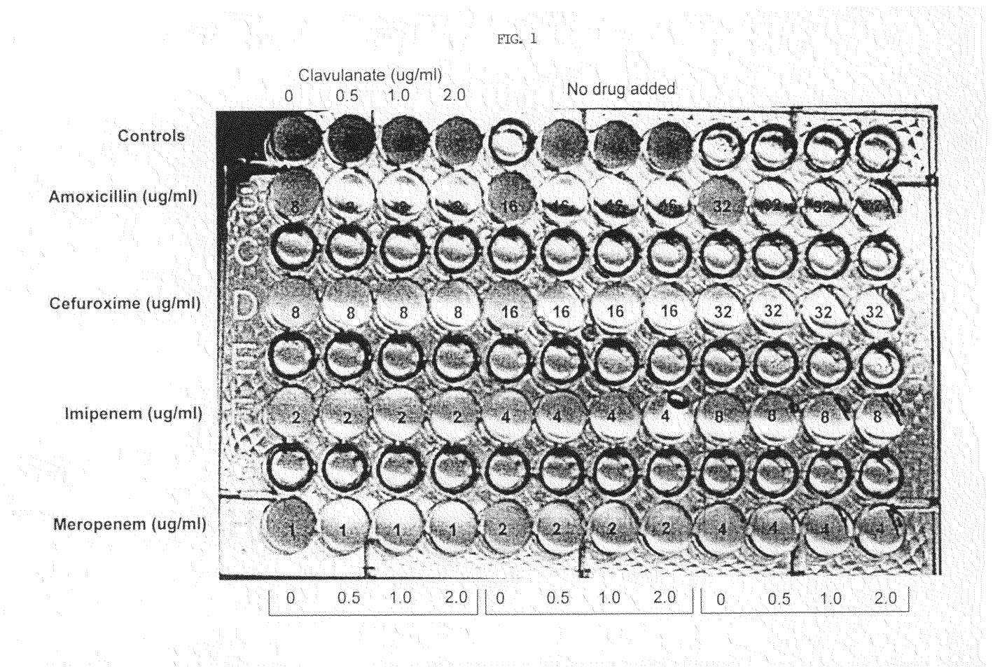

In vitro tests using M. tuberculosis Erdman strain were performed to test the hypothesis that treatment of M. tuberculosis with clavulanate would act to sensitize an organism to β-lactams, and therefore would be an effective new treatment for tuberculosis (FIG. 1).

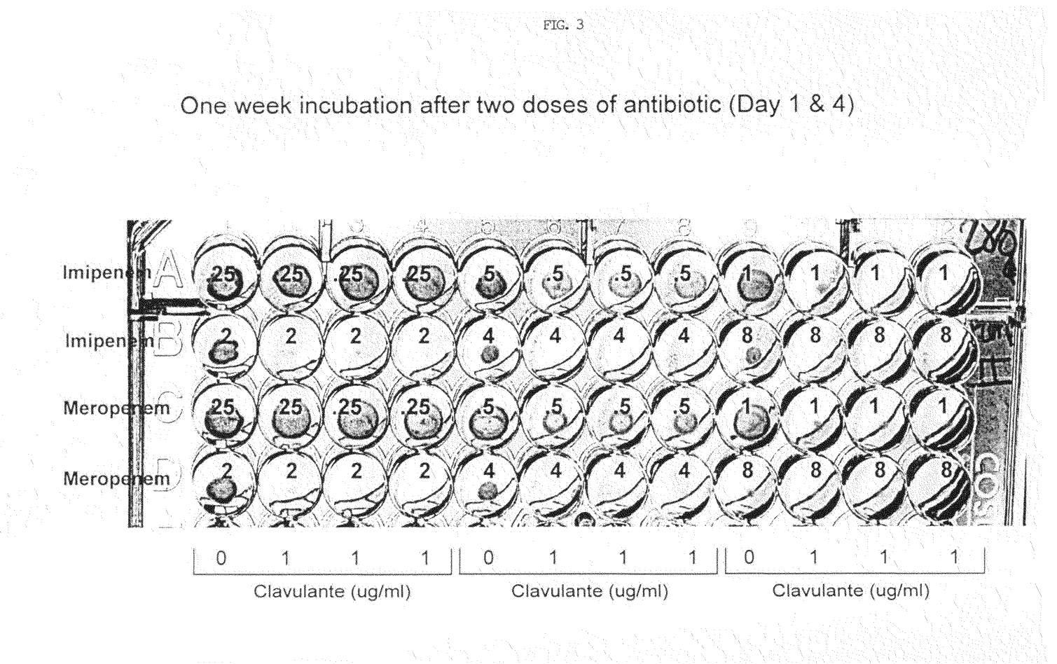

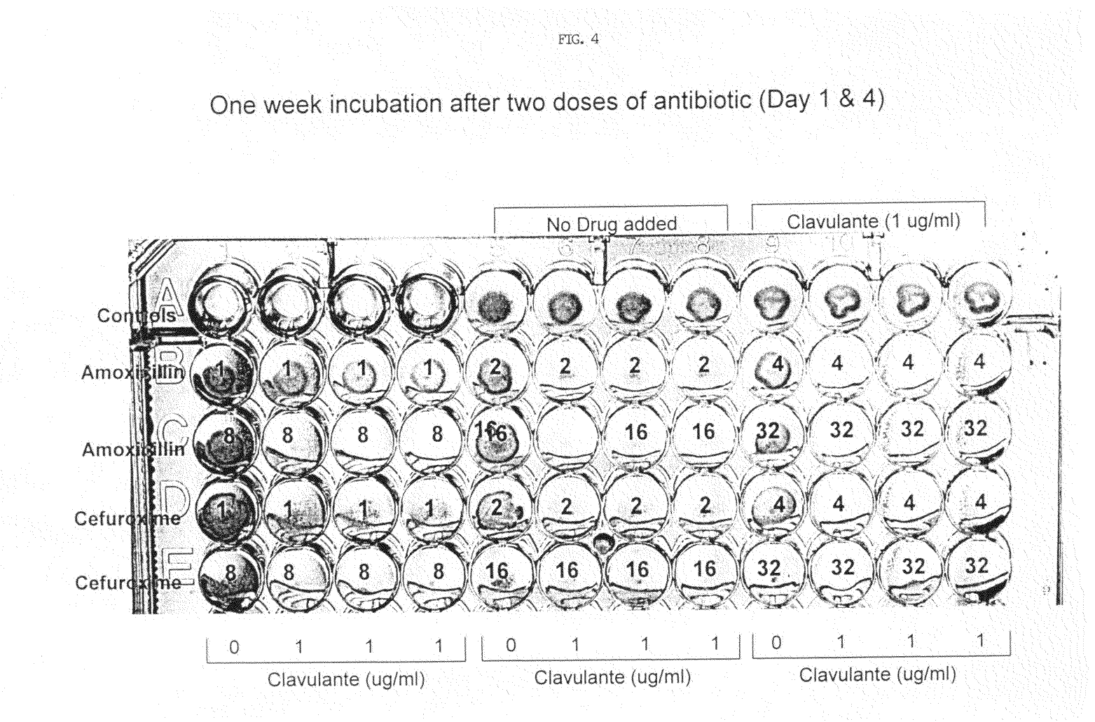

The effects of various concentrations of clavulanate (0.0.5, 1 and 2 μg/ml) on the inhibitory activity of various β-lactams were observed. The selected β-lactams were piperacillin, ceftazidime, amoxicillin, cefuroxime, imipenem, and meropenem. A broth microdilution method with 7H9 broth in 96 well plates was used, and M. tuberculosis growth was observed after 1 week of incubation. β-lactam doses were administered either once during the 1 week incubation period (Day 1), or twice (Days 1 and 4) Concentrations of the β-lactams and clavulanate are indicated on FIGS. 1-4.

### Results and Discussion.

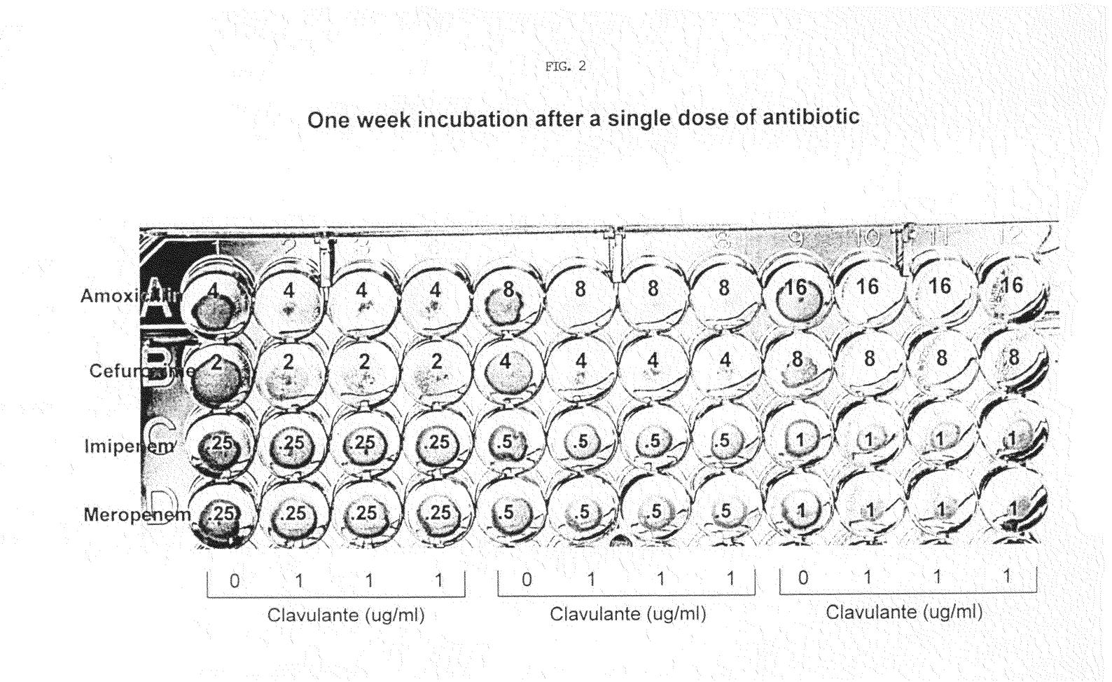

After one week of incubation, no inhibition of M. tuberculosis growth by piperacillin or ceftazidime at any concentration of clavulanate was observed. Modest inhibition of M. tuberculosis growth was observed with 4 μg/ml of amoxicillin and 1-2 μg/ml clavulanate, as well as 2 μg/ml cefuroxime and 2 μg/ml clavulanate. Of the two carbapenems tested (imipenem and meropenem), imipenem showed inhibition of M. tuberculosis growth at 1-2 μg/ml and 0.5-2 μg/ml clavulanate, with the strongest inhibition observed in the plates with the highest concentrations of imipenem. No M. tuberculosis growth was observed in plates containing 1 μg/ml meropenem in combination with 1 μg/ml clavulanate. See FIGS. 2-4.

Based on results of the above experiments, it is shown that the administration of meropenem, imipenem, or cefuroxime in combination with clavulanate is an effective new treatment for tuberculosis.

### Example 2

The minimum inhibitory concentration (MIC) values of M. tuberculosis H37Rv in 7H9 medium at 37° C. by penicillins, cephalosporins, and carbapendems in the absence and presence of 2.5 μg ml⁻¹ clavulanate was determined. The addition of calvulanate had only a modest effect on the MIC values of ampicillin and amoxicillin but a significant effect on the MIC values of cephalothin, imipenem, and meropenem (data not shown). On the basis of the low MIC value of meropenem in the presence of clavulanate (0.32 μg ml⁻¹) and its low rate of hydrolysis by BlaC, this carbapenem was selected for detailed analysis. When various combinations of meropenem and clavulanate were added daily for 5 consecutive days to cultures of M. tuberculosis Erdman under aerobic growth conditions, the colony-forming units per milliliter (CFU ml⁻¹) dropped rapidly until complete sterilization was obtained after 9 to 12 days (data not shown). Although the number and identity of the cell wall cross-linking transpeptidase targets of meropenem in M. tuberculosis are not known, it is clear that a combination of calvulanate and meropenem rapidly sterilizes actively growing aerobic cultures of M. tuberculosis.

An important problem in tuberculosis therapy is the phenotypic drug resistance of populations of organisms that are in a nonreplicative state, termed “persistence” (13, 14). The M. tuberculosis L,D-transpeptidase has recently been reported to be a target for carbapenems, and this enzyme is thought to be expressed as the organism enters into the persistent state, with corresponding changes in the nature of peptidoglycan cross-linking (15). There are several in vitro models of this state, but the most widely used is the Wayne model (16), where organisms that are grown in sealed tubes enter into a viable but nonreplicative (NRP) state after 2 weeks because of consumption of available oxygen. Combinations of clavulanate and meropenem were tested for their ability to sterilize organisms in this state. Drug combinations were added to NRP2 cultures within an anaerobic chamber, and cellular viability was assessed 1 week and 2 weeks later by measuring intracellular adenosine triphosphate (ATP) concentrations as well as CFUs. All clavulanate β-lactam combinations were effective in reducing viability, but the decrease was more pronounced with the two carbapendems, imipenem and meropehem, than with amoxillin and cefuroxime (data not shown). In the case of the clavulanate-meropenem combination, there was observed more than a log kill over a 2-week exposure time, comparable to metronidazole, which was used as a control compound. The combination of clavulanate with β-lactams, especially meropenem, was also tested for the ability to inhibit the growth of extensively drug-resistant (XDR) clinical strains of M. tuberculosis. Thirteen clinical isolates exhibiting the XDR phenotype were tested (17). Clavulanate was used at a concentration of 2.5 μg ml⁻¹, and the MIC values of these strains for meropenem were determined. The susceptibility of these strains was experimentally indistinguishable from that determined for H37Rv and the Erdman strain, that is, ≦1 μg ml⁻¹ (data not shown). In contrast, substantial variability of MIC values to ampicillin, cephalothin, and imipenem was observed for the same strains (data not shown). The clavulanate-meropenem combination is thus equally effective against both susceptible and XDR strains.

### REFERENCES

1. Gandhi, N. R., et al. “Extensively drug-resistant tubercolosis as a cause of death in patients co-infected with tuberculosis and HIV in a rural area of South Africa” Lancet 368:1575-1580 (2006).
2. Goffin, C. and Ghuysen, J. M. “Multimodular penicillin-binding proteins: An enigmatic family of orthologs and paralogs” Microbiol. Mol. Biol. Rev. 62:1079-1093 (1998).
3. Flores, A. R., et al. “Genetic analysis of the β-lactamases of Mycobacterium tuberculosis and Mycobacterium smegmatis and susceptibility to β-lactam antibiotics” Microbiology 151:521-532 (2005).
4. Meroueh, S. O., et al. “Ab initio QM/MM study of class A β-lactamase acylation: Dual participation of Glu166 and Lys73 in a concerted base promotion of Ser70” J. Am. Chem. Soc. 127:15397-15407 (2005).
5. Escobar, W. A., et al. “Site-directed mutagenesis of glutamate-166 in β-lactamase leads to a branched path mechanism” Biochemistry 33:7619-7626 (1994).
6. Damblon, C. et al. “The catalytic mechanism of β-lactamases: NMR titration of an active-site lysine residue of the TEM-1 enzyme” Proc. Natl. Acad. Sci. U.S.A. 93:1747-1752 (1996).
7. Adachi, H., et al. “Site-directed mutants, at position 166, of RTEM-1 β-lactamase that form a stable acyl-enzyme intermediate with penicillin” J. Biol. Chem. 266:613-619 (1991).
8. Gibson, R. M., et al. “Site-directed mutagenesis of β-lactamase I. Single and double mutants of Glu-166 and Lys-73” Biochem. J. 272:613-619 (1990).
9. U.S. Pat. No. 5,726,170 for Callewaert issued Mar. 10, 1998.
10. U.S. Pat. No. 6,048,977 for Cole, et al. issued Apr. 11, 2000.
11. U.S. Pat. No. 6,051,703 for Cole, et al. issued Apr. 18, 2000.
12. U.S. Pat. No. 5,994,534 for Capuder issued Nov. 30, 1999.
13. J. E. Gomez, J. D. McKinney, Tuberculosis (Edinburgh) 84, 29 (2004).
14. Y. Zhang, Front. Biosci. 9, 1136 (2004).
15. M. Lavollay, et al., J. Bacteriol. 190, 4360 (2008).
16. L. G. Wayne, L. G. Hayes, Infect. Immun. 64, 2062 (1996).
17. C. Y. Jeon, et al., Clin. Infect. Dis. 46, 42 (2008).

1. A method for treating tuberculosis in a subject comprising administering to the subject an amount of carbapenem and clavulanic acid or salt thereof effective to treat tuberculosis.

2. The method of claim 1, wherein the tuberculosis is multidrug-resistant tuberculosis.

3. The method of claim 1, wherein the tuberculosis is extensively multidrug-resistant tuberculosis.

4. The method of claim 1, wherein the subject is human.

5. The method of claim 1, wherein the carbapenem and clavulanic acid or salt thereof are formulated for parenteral administration or oral administration.

6. The method of claim 5, wherein the parenteral administration is intramuscular, intrathecal, subcutaneous, intraperitoneal, or intravenous bolus injection, or intravenous infusion.

7. The method of claim 1, wherein the carbapenem and clavulanic acid or salt thereof are administered in the same pharmaceutical formulation.

8. The method of claim 1, wherein the carbapenem and clavulanic acid or salt thereof are administered in different pharmaceutical formulations.

9. The method of claim 1, wherein the carbapenem is meropenem, imipenem, ertapenem, faropenem, doripenem, or panipenem.

10. The method of claim 9, wherein the carbapenem is meropenem.

11. The method of claim 10, wherein the amount of meropenem effective to treat tuberculosis is about 1500 mg/day to about 3000 mg/day.

12. The method of claim 9, wherein the carbapenem is imipenem.

13. The method of claim 12, wherein the therapeutically effective amount of imipenem is about 1000 mg/day to 2000 mg/day.

14. The method of claim 1, wherein the therapeutically effective amount of clavulanic acid is about 50 mg/day to about 500 mg/day.

15. The method of claim 1, wherein the clavulanic acid is clavulanate.

16. The method of claim 15, wherein the therapeutically effective amount of clavulanate is about 50 mg/day to about 500 mg/day.

17. A method for treating tuberculosis in a subject comprising administering to the subject an amount of cefuroxime and clavulanic acid or salt thereof effective to treat tuberculosis.

18-28. (canceled)

29. A pharmaceutical composition comprising therapeutically effective amounts of carbapenem and clavulanic acid or salt thereof, and a pharmaceutically acceptable carrier.

30-39. (canceled)

40. A pharmaceutical composition comprising a therapeutically effective amount of cefuroxime and clavulanic acid or salt thereof, and a pharmaceutically acceptable carrier.

41-46. (canceled)

47. A method for manufacturing the pharmaceutical composition of claim 29 comprising formulating therapeutically effective amounts of carbapenem and clavulanic acid or salt thereof with a pharmaceutically acceptable carrier.

48-57. (canceled)

58. A method for manufacturing the pharmaceutical composition of claim 40 comprising formulating therapeutically effective amounts of cefuroxime and clavulanic acid or salt thereof with a pharmaceutically acceptable carrier.

59-72. (canceled)
